(19) United States
(12) Patent Application Publication
(10) Pub. No.: US 2014/0198883 A1
(43) Pub. Date: Jul. 17, 2014

(54) Systems and Methods for Highly Accurate and Efficient Pulse Prediction

(71) Applicant: Board of Governors for Higher Education, State of Rhode Island and Providence Plantations, Providence, RI (US)

(72) Inventors: Steven Kay, Middletown, RI (US); Andrew O'Shea, Pawtucket, RI (US)

(73) Assignee: Board of Governors for Higher Education, State of Rhode Island and Providence Plantations, Providence, RI (US)

(21) Appl. No.: 14/136,459

(22) Filed: Dec. 20, 2013

Related U.S. Application Data
(60) Provisional application No. 61/740,020, filed on Dec. 20, 2012.

(51) Int. Cl. A61B 5/00; A61B 5/024; H04L 7/033

(52) U.S. Cl. CPC A61B 5/7246; H04L 7/033; A61B 5/024
USPC 375/342

(57) ABSTRACT

A method is disclosed for predicting a pulse periodicity in a signal. The method includes the steps of receiving a signal that includes a component that is periodic having an unknown periodicity and a noise component; comparing the signal to an adjustable reference signal, and varying at least one of the phase and the periodicity of the reference signal until a best fit match is obtained between the signal and the reference signal.

### PRIORITY

The present application claims priority to U.S. Provisional Patent Application Ser. No. 61/740,020 filed Dec. 20, 2012, which is hereby incorporated by reference in its entirety.

### BACKGROUND

Heart rate monitors generally fall into two categories. In the first case they attempt to detect the QRS complexes using knowledge of the signal and then calculate the time between the signals to be used to estimate the period, with the heart rate given by the reciprocal. A second approach, which does not require signal knowledge, is to use the peak location of a computed autocorrelation function, but does assume that the signal stays relatively constant over several pulse periods.

The first approach requires exact knowledge of the signal, and noise that can be modeled as white Gaussian noise since typically a matched lter is used. The second approach only works well in relatively noiseless environments. Unfortunately, for heart rate estimation based on the output of a skin contact sensor of a person that is exercising neither of these requirements are satisfied. The available waveform contains a QRS complex that is unknown a priori (depends on the person), may change within several heart beats, and is embedded in noise and interference that is non-stationary, non-Gaussian, and with samples that need not be independent. The method described herein does not require any of these restrictive assumptions to be made.

There remains a need therefore, for providing pulse prediction and estimation that may be efficiently and economically implemented in a high noise environment.

### SUMMARY

In accordance with an embodiment, the invention provides a method for predicting a pulse periodicity in a signal. The method includes the steps of receiving a signal that includes a component that is periodic having an unknown periodicity and a noise component; comparing the signal to an adjustable reference signal, and varying at least one of the phase and the periodicity of the reference signal until a best fit match is obtained between the signal and the reference signal.

In accordance with another embodiment, the invention provides a method of estimating a heart pulse rate in a subject. The method includes the steps of receiving a signal that is representative of a heart rate and includes a component that is periodic having an unknown periodicity and a noise component; identifying instances of peak pulse power in the signal; comparing the instances of pulse peak power of the signal to an adjustable reference signal, and varying at least one of the phase and the periodicity of the reference signal until a best fit match is obtained between the signal and the reference signal.

In accordance with a further embodiment, the invention provides a system for estimating a heart pulse rate in a subject. The system includes an input port for receiving a signal that is representative of a heart rate and includes a component that is periodic having an unknown periodicity and a noise component; an identification module for identifying instances of peak pulse power in the signal; a comparison module for comparing the instances of pulse peak power of the signal to an adjustable reference signal, and an adjustment module for varying at least one of the phase and the periodicity of the reference signal until a best fit match is obtained between the signal and the reference signal.

### BRIEF DESCRIPTION OF DRAWINGS

The following description may be further understood with reference to the accompanying drawings in which:

FIG. 1 shows an illustrative schematic view of a process in accordance with an embodiment of the present invention;

FIG. 2 shows an illustrative graphical representation of modeling assumptions for power variation with time; and

FIGS. 3A and 3B show illustrative timing diagrams in a system in accordance with an embodiment of the present invention.

### DETAILED DESCRIPTION

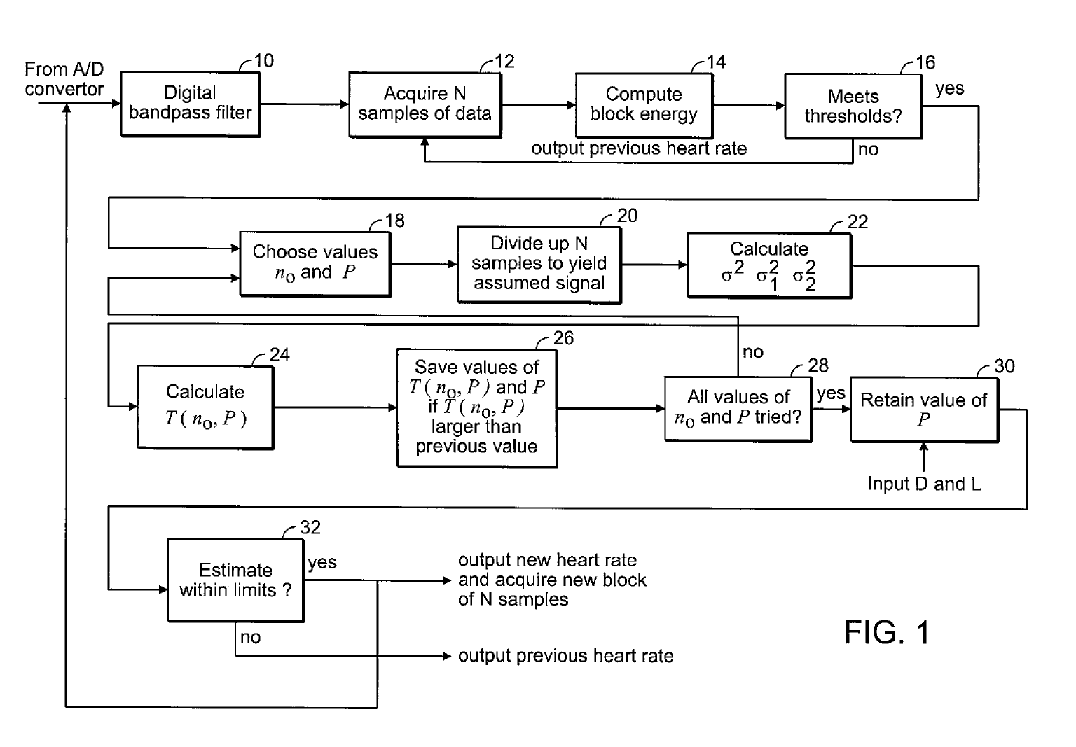

Systems and methods of certain embodiments of the present invention rely on two salient features of a periodic signal embedded in noise and/or interference. The first feature is that when the signal is present, the power increases and secondly, this power increase due to the presence of a signal occurs at a periodic interval. No other modeling assumptions are needed. As such, its performance is robust to the many different effects present due to the sensor, source of the signal, etc. There is no training and/or calibration required prior to its use by an individual. A general diagram of the invention is shown in FIG. 1 and is explained below.

As shown in FIG. 1, in a first step, the system receives an input signal from an A/D converter at a digital bandpass filter (10), which then communicates to a module that acquires N samples of date (12). In certain embodiments, the system may receive data over a limited specified period of time. The system then computes the block energy (14) and determines whether a threshold has been met (16). The system then chooses values of n₀ and P (18), and then divides up N samples to yield an assumed signal (20). The system then calculates (22). The system then calculates T(n₀,P) (24), and then sets values of T(n₀,P) and P if T(n₀,P) is larger than the previous value (26). The system then determines whether all values of n₀ and P have been tried, and if not returns to the module of choosing values of n₀ and P (18). If all values of n₀ and P have been tried, the system retains the value of P (30) and the determines whether the estimate is within set limits (32). If so, a new heart rate is output and a new block of N samples if acquired.

### Example 1

### Raw Estimator of Heart Rate

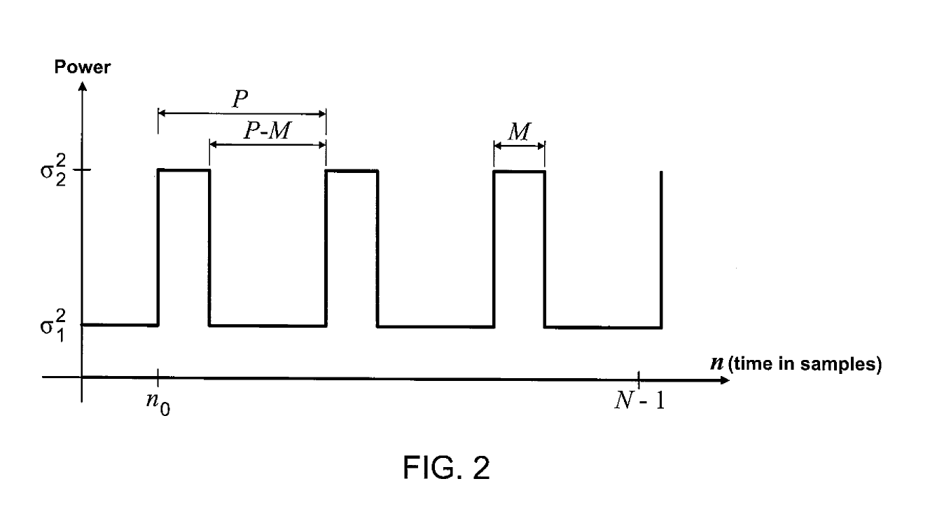

The model assumes that the variation of the power of the acquired signal versus time in samples is as shown in FIG. 2. This represents a block of the data, typically a few seconds, after it has been sampled by an A/D convertor, input to a CPU, and digitally bandpass filtered by an FIR filter. The symbols displayed in FIG. 2 are: n₀ is the starting time of the first pulse, M is the width of the pulse, and P is the period of the power variation. The power changes from σ₁² to σ₂² (σ₂²>σ₁²) when a pulse is present. The range of possible values of the period is Pₘᵢₙ≦P≦P_{max□} and for the start time it is 0≦n₀≦P−1. The pulse width M is assumed known as is P_{min □} and P_{max□} while all the other parameters {σ₁², σ₂², n₀, P} are not known in advance. The device estimates all the unknown parameters but pays particular attention to P, which yields the pulse rate of

$$\frac{1}{p}$$

cycles/sample. This is then converted to BPM for the heart rate application. For a sampling rate of Fs samples/sec of the A/D convertor the final heart rate is given by

$$BPM = \frac{60 {F}_{s}}{P} .$$

The period P is estimated by performing a numerical maximization of a function T(n₀, P) over its allowable values. The function is defined by

$$\begin{matrix}T ( {n}_{0} , P ) = \{ \begin{matrix}N log {\hat{σ}}^{2} - {N}_{1} log {\hat{σ}}_{1}^{2} - {N}_{2} log {\hat{σ}}_{2}^{2} & {\hat{σ}}_{2}^{2} > {\hat{σ}}_{1}^{2} \\ 0 & {\hat{σ}}_{2}^{2} \leq {\hat{σ}}_{1}^{2}\end{matrix} \\ where & ( 1 ) \\ {\hat{σ}}^{2} = 2 {( \frac{1}{N} {\sum }_{n = 0}^{N - 1} \vert x [ n ] \vert )}^{2} & ( 2 ) \\ {\hat{σ}}_{1}^{2} = 2 {( \frac{1}{{N}_{1}} {\sum }_{i ∈ {S}_{1}} \vert {x}_{1} [ i ] \vert )}^{2} & ( 3 ) \\ {\hat{σ}}_{2}^{2} = 2 {( \frac{1}{{N}_{2}} {\sum }_{i ∈ {S}_{2}} \vert {x}_{2} [ i ] \vert )}^{2} . & ( 4 )\end{matrix}$$

The data samples needed to compute t(n₀, P) are z[n], which are the samples of the entire block of samples under consideration (see FIG. 2). The data samples x₁[n] and x₂[n] are the samples corresponding to those between the pulses and those corresponding to the pulse intervals, respectively, for an assumed value of (n₀, P). The set of samples for s₁[n] is denoted by S₁ and the set of samples for x₂[n] is denoted by S₂. The total number of samples is N for x[n], N₁ for x₁[n], and N₂ for x₂[n]. The function must be computed for Pₘᵢₙ≦P≦P_{max□}and for each value of P, it must also be computed for 0≦n₀≦P−1, resulting in a two-dimensional matrix computation. The value of (n₀, P) that yields the maximum value of T(n₀, P) is designated as the estimate of n₀ and P, with P being the parameter of interest for the heart rate application. The period estimate is calculated for a block of data T seconds in length, where typically T=3, but could be otherwise. The blocks of data are overlapped by a certain percentage with a typical percentage being 75%, but could be otherwise. For these choices an estimate is computed every ¾ second. Not all estimates are reported to the user. If the estimate of P is deemed to be inaccurate, the previous estimate is maintained.

### Example 2

### Reported Estimate of Heart Rate—Continuous Monitoring

The device has two modes of operation: continuous and intermittent. Considering the former, the new estimate is reported if the new period estimate is sufficiently close to previous estimates. The reporting rule is that the new estimate is reported if the period difference is less than D samples from all of the previous L estimates. Otherwise, the previous estimate is maintained and reported. Typical values for the reporting parameters are D=10 and L=2 at a sampling rate of

$${F}_{s} = 360 \frac{samples}{sec □} .$$

The choice of these parameters relates to the tradeoff between obtaining a heart estimate with a given amount of delay and the accuracy of that heart rate estimate.

### Example 3

### Reported Estimate of Heart Rate—Intermittent Monitoring

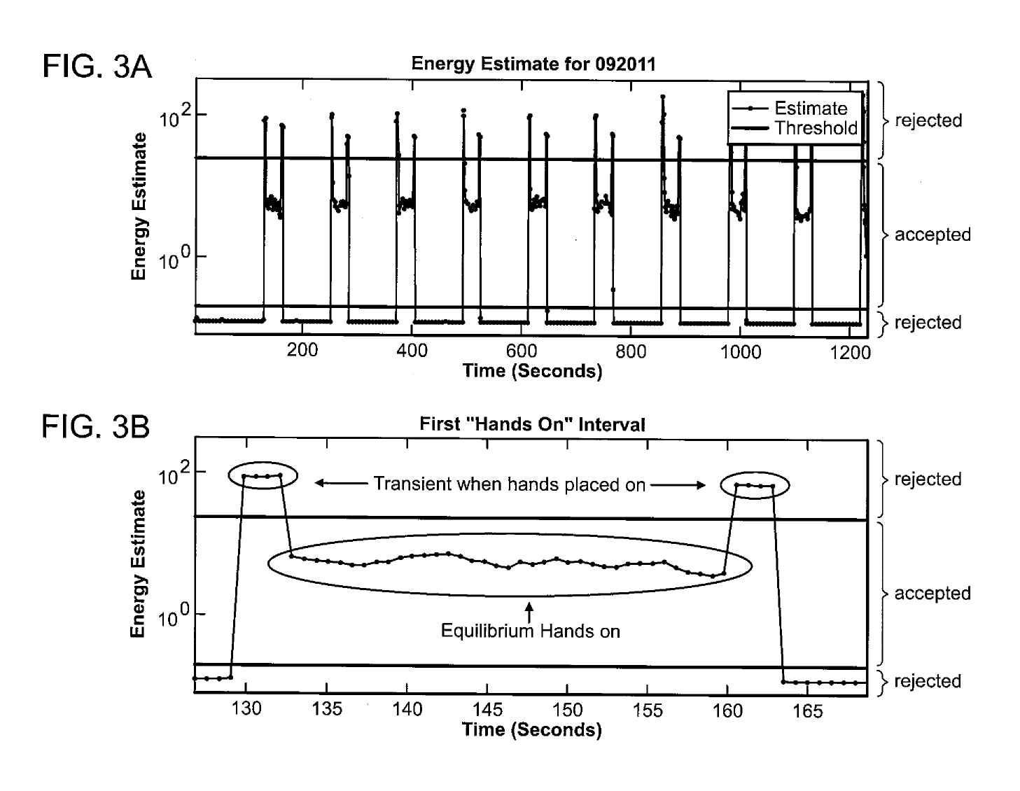

For applications where the heart rate estimate is only required at certain times, such as is typical for exercise equipment in which the user places his/her hands on a contact sensor, the reporting logic has an additional step. As shown in FIG. 3 the energy of the acquired heart rate signal obtained through a hand-held contact exhibits a sharp transient. To determine the interval over which the user's hands are actually on the contact, indicating a desired heart rate reading, two thresholds are used. The lower threshold indicates the hands are off since the energy does not exceed the threshold and so the estimate is not reported. Likewise, once the hands are applied there is a sharp transient. A second threshold is used to determine when the transient has subsided. When the energy is less than this second threshold, the estimate is reported. The energy is estimated using

$$\hat{ɛ} = {\sum }_{n = 0}^{N - 1} {x}^{2} [ n ]$$

and is computed for the samples within the current block. The thresholds are adjustable and are set by the sensing characteristics of the device employing the heart rate monitor.

Modifications may be made to the system to reduce the computational complexity in C programming. The performance loss due to these modifications are minimal. First, fixed-point numbers may be used instead of floating-point numbers. Second, a look-up table may be used for logarithm instead of the log function itself. And third, a non-uniform sampling method may be used instead of searching every P from Pₘᵢₙ to Pₘₐₓ.

The look-up table for logarithm and the non-uniform sampling grid of P may be determined off-line and stored in the memory for later use. The MATLAB code to generate the look-up table may be:

x=(0:65535)′;

y=1000*log (x);

y(1)=0;

z=int16(y);

fileID=fopen(‘LUT.txt’,‘w’);

fprintf(fileID,‘%d ,’,z);

fclose(fileID);

The non-uniform sampling method may be implemented as follows. The original searches for P (period in samples) may be from Pₘᵢₙ=120 (200 bpm) to Pₘₐₓ =400 (60 bpm). Therefore, the original searches over 400−120+1=2817 candidate P's. For the non-uniform sampling, it is desired to search over less points (e.g., L=140) of P. This would reduce the computational complexity by a factor of 281/140≈2 (with some loss of performance, which is negligible in practice). Using a uniform grid however, (i.e. P=120:400−120 L−1:400) would not be a good choice. This is because the Fisher Information Matrix of P and nₒ is shown to be:

$$I ( {n}_{0} , P ) = \frac{N {τ}^{3}}{24 {σ}^{4}} [ \begin{matrix}\frac{T}{P} & \frac{1}{2} {( \frac{T}{P} )}^{2} \\ \frac{1}{2} {( \frac{T}{P} )}^{2} & \frac{1}{3} {( \frac{T}{P} )}^{3}\end{matrix} ]$$

As a result, the variance of ̂ P depends on P. With the transformation

$$γ ( P ) = \frac{1}{\sqrt{P}}$$

which has the property that

[I(n0,γ)]₂₂=cT³

The Fisher Information therefore of γ does not depend on P, and we wish to search over a uniform grid on γ. Then we transform y back to P by P=1 γ2 , and get a non-uniform grid on P. The MATLAB code to generate the non-uniform sampling grid of P is

Pmin=120;

Pmax=400;

L=140;

thetamax=1/sqrt(Pmin);

thetamin=1/sqrt(Pmax);

% Variance-stabilizing transformation

theta=thetamin:(thetamax-thetamin)/(L-1):thetamax;

P=1./(theta.̂2);

P=round(P);

fileID=fopen(‘NonUniformP.txt’,‘w’);

fprintf(fileID,'%d ,',P);

fclose(fileID);

A method is therefore disclosed of measuring the heart rate of individuals based on the voltage potential across selected skin points is described. The device is able to extract highly accurate estimates for sensing devices that are prone to noise due to effects such as poor sensor contact, muscle noise, and other undesirable artifacts that obscure the QRS complex. The method uses a unique model for the signal waveform that is appropriate in these cases. The output of the device is a reading of heart rate in beats per minute (BPM) that can be displayed either continuously or at intermittent times and either displayed for immediate reading or stored for future use. The device is capable of measuring and outputting the rate of any periodic signal when obscured by noise, whether the signal form is known or not. It does not require any training by a potential user before actual operation. The heart rate monitor application as described herein serves as an indication of it utility and implementation.

In accordance with various embodiments therefore, the invention provides a method of estimating pulse prediction of a signal that includes periodic pulses embedded in noise and interference, wherein the signal is selected from a group consisting of electrical sensing, optical sensing, or any remote sensing device. The signal does not require explicit knowledge of the pulse waveform. The method does not require time synchronization of the pulse starting times, and the period of the signal may be robust with respect to gain changes of the acquired signal. The method may include estimating the starting time of a periodic signal, and may identify and edit out poor estimates. The statistical signal processing model accurately predicts the salient features of a skin-contact acquired EKG signal, and the model does not need to make the usual signal and noise assumption that they are additive in voltage, only in power. The method determines the presence of skin-contact on an electrode. The system provides a means to trade of the speed of period acquisition and the accuracy of the period estimate, and the method is robust with respect to the corrupting noise statistical characteristics, in particular, its probability density function and its power spectral density. The system and method also do not require training of any kind to acquire any information about a particular user prior to its operation

Those skilled in the art will appreciate that numerous modifications and variations may be made to the above disclosed embodiments without departing from the spirit and scope of the present invention.

1. A method of predicting a pulse periodicity in a signal, said method comprising the steps of receiving a signal that includes a component that is periodic having an unknown periodicity and a noise component; comparing the signal to an adjustable reference signal, and varying at least one of the phase and the periodicity of the reference signal until a best fit match is obtained between the signal and the reference signal.

2. The method as claimed in claim 1, wherein said method further includes the step of identifying instances of peak pulse power in the signal, and wherein said step of comparing the adjustable reference signal to the signal involves comparing the adjustable reference signal to the instances of pulse peak power of the signal.

3. The method as claimed in claim 1, wherein said method further includes the step of providing an output indication of the period of the component of the signal that is periodic.

4. The method as claimed in claim 1, wherein the signal and the adjustable reference signal are not synchronized with each other prior to performing the steps of claim 1.

5. The method as claimed in claim 1, wherein the signal represents a heart rate of a subject.

6. A method of estimating a heart pulse rate in a subject, said method comprising the steps of receiving a signal that is representative of a heart rate and includes a component that is periodic having an unknown periodicity and a noise component; identifying instances of peak pulse power in the signal; comparing the instances of pulse peak power of the signal to an adjustable reference signal, and varying at least one of the phase and the periodicity of the reference signal until a best fit match is obtained between the signal and the reference signal.

7. The method as claimed in claim 6, wherein said method further includes the step of providing an output indication of the period of the component of the signal that is periodic.

8. The method as claimed in claim 6, wherein the signal and the adjustable reference signal are not synchronized with each other prior to performing the steps of claim 1.

9. A system for estimating a heart pulse rate in a subject. The system includes an input port for receiving a signal that is representative of a heart rate and includes a component that is periodic having an unknown periodicity and a noise component; an identification module for identifying instances of peak pulse power in the signal; a comparison module for comparing the instances of pulse peak power of the signal to an adjustable reference signal, and an adjustment module for varying at least one of the phase and the periodicity of the reference signal until a best fit match is obtained between the signal and the reference signal.

10. The system as claimed in claim 9, wherein said system further includes an output port for providing an output indication of the period of the component of the signal that is periodic.

11. The system as claimed in claim 9, wherein the signal and the adjustable reference signal are not synchronized with each other prior receiving the signal at the input port.
